(19) United States
(12) Patent Application Publication
(10) Pub. No.: US 2004/0224409 A1
(43) Pub. Date: Nov. 11, 2004

(54) Recombinant adenoviruses coding for brain-derived neurotrophic factor (BDNF)

(76) Inventors: Laurent Pradier, Paris (FR); Pascal Barneoud, Choisy Le Roi (FR); Pia Delaere, Paris (FR); Michel Perricaudet, Ecrosnes (FR); Emmanuelle Vigne, Ivry Sur Seine (FR)

Correspondence Address: WILEY, REIN & FIELDING, LLP, ATTN: PATENT ADMINISTRATION, 1776 K. STREET N.W., WASHINGTON, DC, 20006, US

(21) Appl. No.: 10/742,920

(22) Filed: Dec. 23, 2003

Related U.S. Application Data
(63) Continuation of application No. 08/716,209, filed on Oct. 9, 1996, now abandoned.
Application No. 08/716,209 is a 371 of international application No. PCT/FR95/00250, filed on Mar. 2, 1995.
(63) Continuation-in-part of application No. 08/403,868, filed on Apr. 28, 1995, now abandoned.
Application No. 08/403,868 is a 371 of international application No. PCT/EP93/02519, filed on Sep. 17, 1993.

(30) Foreign Application Priority Data
Mar. 18, 1994 (FR) FR94/03191
Sep. 25, 1992 (EP) EP92-402644.6

(51) Int. Cl. C12N 15/861; C12N 7/01

(52) U.S. Cl. 435/456; 435/235.1

(57) ABSTRACT

Recombinant adenoviruses comprising a heterologous DNA sequence coding for brain-derived neurotrophic factor (BDNF), preparation thereof, and use thereof for treating and/or preventing degenerative neurological diseases.

The present invention relates to recombinant vectors of viral origin and to their use for the treatment and/or prevention of neurodegenerative diseases. More particularly, it relates to recombinant adenoviruses containing a DNA sequence encoding brain-derived neurotrophic factor (BDNF, brain-derived neurotrophic factor). The invention also relates to the preparation of these vectors, to pharmaceutical compositions containing them and to their therapeutic use, especially in gene therapy.

Neurodegenerative diseases represent a substantial part of health expenditure in Western countries, a part which is increasingly rising as a result of the ageing of the population. As examples of these conditions, there may be mentioned especially Alzheimer's disease, Parkinson's disease, Huntington's chorea, amyotrophic lateral sclerosis and the like. The pathological signs and the aetiology of these diseases are quite varied, but all these diseases result from a gradual loss of neuron cells in the central nervous system, sometimes in highly localized structures such as the black substance in Parkinson's disease. Although some palliative pharmacological treatments-are already available, their effects are relatively limited. The present invention describes a new, particularly advantageous, therapeutic approach for the treatment of these diseases. More particularly, the present invention describes vectors which make it possible to promote directly the survival of the neuron cells 5′ implicated in these pathologies, by the effective and localized expression of certain trophic factors.

Trophic factors are a class of molecules having properties for stimulating neuritic growth or the survival of nerve cells. The first factor possessing neurotrophic properties, NGF (Nerve Growth Factor), was characterized about forty years ago (for a review, see Levi-Montalcini and Angelleti, Physiol. Rev. 48 (1968) 534). It was only recently that other neurotrophic factors were identified, and especially the brain-derived neurotrophic factor (BDNF) (Thoenen, Trends in NeuroSci. 14 (1991) 165). BDNF is a protein with 118 amino acids and a molecular weight of 13.5 kD. In vitro, BDNF stimulates the formation of neurites and the survival in culture of the ganglionic neurons of the retina, the motoneurons of the spinal cord, the cholinergic neurons of the septum as well as the dopaminergic neurons of the mesencephalon (review by Lindsay in Neurotrophic Factors, Ed, (1993) 257, Academic Press). However, although its properties are advantageous, the therapeutic application of BDNF is confronted with various obstacles. In particular, the absence of bioavailability of BDNF limits any therapeutic use. Moreover, there is no effective means allowing BDNF to be delivered in a durable and localized manner to certain desired regions of the body. Finally, it is essential that the BDNF-delivered is active and can exert a therapeutic activity in vivo.

The present invention provides a particularly advantageous solution to these problems. The present invention indeed consists in the development of particularly effective vectors to deliver in vivo and locally, therapeutically active quantities of BDNF. In copending Application No. PCT/EP93/02519, it has been shown that adenoviruses could be used for transferring genes in vivo into the nervous system. The present invention relates to new constructs which are particularly adapted and effective for transferring a specific gene into the nervous system. More particularly, the present invention relates to a recombinant adenovirus comprising a DNA sequence encoding brain-derived neurotrophic factor (BDNF), to its preparation, and to its use for the treatment and/or prevention of neurodegenerative diseases.

The Applicant has now shown that it is possible to construct recombinant adenoviruses containing a sequence encoding BDNF, to administer these recombinant adenoviruses in vivo, and that this administration allows a stable and localized expression of therapeutically active quantities of BDNF in vivo, and in particular in the nervous system, and without cytopathological effect. The particularly advantageous properties of the vectors of the invention stem especially from the construction used (defective adenovirus, deleted of certain viral regions), the promoter used for the expression of the sequence encoding BDNF (preferably viral or tissue-specific promoter), and the methods for administering the said vector, allowing the efficient expression, and in the appropriate tissues, of BDNF. The present invention thus provides viral vectors which can be used directly in gene therapy, which are particularly adapted and efficient for directing the expression of BDNF in vivo. The present invention thus offers a particularly advantageous new approach for the treatment and/or prevention of neurodegenerative diseases.

A first subject of the invention therefore consists in a defective recombinant adenovirus comprising a DNA sequence encoding brain-derived neurotrophic factor (BDNF) or a derivative thereof.

The subject of the invention is also the use of such a defective recombinant adenovirus for the preparation of a pharmaceutical composition intended for the treatment or prevention of neurodegenerative diseases.

The brain-derived neurotrophic factor (BDNF) produced within the framework of the present invention may be human BDNF or animal BDNF. The DNA sequence encoding human BDNF and rat BDNF has been cloned and sequenced (Maisonpierre et al., Genomics 10 (1991) 558), as well as especially the sequence encoding pig BDNF (Leibrock et al., Nature 341 (1989) 149). Prior to their incorporation into an adenovirus vector according to the invention, these sequences are advantageously modified, for example by site-directed mutageneses, in particular for the insertion of appropriate restriction sites. The sequences described in the prior art are indeed not constructed for use according to the invention, and prior adaptations may prove necessary, in order to obtain substantial expressions (see Example 1.2.). Within the framework of the present invention, it is preferable to use a DNA sequence encoding human brain-derived neurotrophic factor (hBDNF). Moreover, as indicated above, it is also possible to use a construct encoding a derivative of BDNF, in particular a derivative of human BDNF. Such a derivative comprises for example any sequence obtained by mutation, deletion and/or addition compared to the native sequence, and encoding a product conserving at least one of the biological properties of BDNF (trophic and/or differentiator effect). These modifications can be carried out by techniques known to persons skilled in the art (see general molecular biology techniques below and Example 2). The biological activity of the derivatives thus obtained can then be easily determined, as indicated especially in Example 3. The derivatives according to the invention can also be obtained by hybridization from nucleic acid libraries, using as probe the native sequence or a fragment thereof.

These derivatives are especially molecules having a higher affinity for their binding sites, sequences permitting an enhanced expression in vivo, molecules having greater resistance to proteases, molecules having greater therapeutic efficacy or fewer side effects, or possibly new biological properties.

Among the preferred derivatives, there may be mentioned more particularly natural variants, molecules in which one or more residues have been substituted, derivatives obtained by deletion of regions having no, or little, involvement in the interaction with the binding sites considered or expressing an undesirable activity, and derivatives containing, compared with the native sequence, additional residues, such as for example a secretion signal and/or a joining peptide. In a particularly advantageous manner, the sequence of the present invention encodes the BDNF preceded by the native pro region (pro BDNF).

Moreover, it is particularly important, for a better implementation of the present invention, for the sequence used also to contain a secretion signal which makes it possible to direct the synthesized BDNF in the secretory pathways of the infected cells, so that the synthesized BDNF is released in the extracellular compartments and can activate its receptors. The secretion signal is advantageously the BDNF signal itself. But it may also be a heterologous or even artificial secretion signal.

The DNA sequence encoding the brain-derived neurotrophic factor used within the framework of the present invention may be a cDNA, a genomic DNA (gDNA) or a hybrid construct consisting for example of a cDNA in which one or more introns could be inserted. This may also be synthetic or semisynthetic sequences. It should be noted that in the genomic sequence encoding BDNF, the introns are located in non-coding regions. In a particularly advantageous manner, a cDNA or a gDNA is used. In particular, the use of a gDNA can permit an enhanced expression in human cells.

In a first embodiment of the invention, the adenovirus therefore comprises a cDNA sequence encoding brain-derived neurotrophic factor (BDNF). In another preferred embodiment of the invention, the adenovirus comprises a gDNA sequence encoding brain-derived neurotrophic factor (BDNF). Advantageously, the DNA sequence encodes proBDNF and, preferably, the preproBDNF.

Advantageously, the sequence encoding BDNF is placed under the control of signals permitting its expression in nerve cells. Preferably, they are heterologous expression signals, that is to say signals different from those which are naturally responsible for the expression of GMF-β. They may be in particular sequences responsible for the expression of other proteins, or of synthetic sequences. In particular, they may be promoter sequences of eukaryotic or viral genes. For example, they may be promoter sequences derived from the genome of the cell which it is desired to infect. Likewise, they may be promoter sequences derived from the genome of a virus, including the adenovirus used. In this respect, there may be mentioned for example the E1A, MLP, CMV, RSV-LTR promoters and the like. In addition, these expression sequences can be modified by the addition of activation or regulatory sequences or sequences permitting a tissue-specific expression. It may indeed be particularly advantageous to use expression signals which are active specifically or predominantly in the nerve cells, so that the DNA sequence is expressed and produces its effect only when the virus has actually infected a nerve cell. In this respect, there may be mentioned for example neuron-specific enolase promoters, GFAP promoters and the like.

In a first specific embodiment, the invention relates to a defective recombinant adenovirus comprising a cDNA sequence encoding brain-derived neurotrophic factor (hBDNF) under the control of the RSV-LTR promoter.

In another specific embodiment, the invention relates to a defective recombinant adenovirus comprising a gDNA sequence encoding brain-derived neurotrophic factor (hBDNF) under the control of the RSV-LTR promoter.

The Applicant has indeed shown that the Rous sarcoma virus (RSV) LTR promoter allowed durable and substantial expression of BDNF in the cells of the nervous, especially central nervous, system.

Still in a preferred embodiment, the invention relates to a defective recombinant adenovirus, having a DNA sequence encoding human brain-derived neurotrophic factor (hBDNF) under the control of a promoter allowing predominant expression in the nervous system. A particularly preferred embodiment of the present invention consists in a defective recombinant adenovirus comprising the ITR sequences, a sequence allowing encapsulation, a DNA sequence encoding human brain-derived neurotrophic factor (hBDNF) or a derivative thereof under the control of a promoter allowing predominant expression in the nervous system and in which the E1 gene and at least one of the E2, E4 and L1-L5 genes is non-functional.

The defective adenoviruses according to the invention are adenoviruses which are incapable of replicating autonomously in the target cell. Generally, the genome of the defective adenoviruses used within the framework of the present invention therefore lacks at least the sequences necessary for the replication of the said virus in the infected cell. These regions can be either removed (completely or partly), or rendered non-functional, or substituted by other sequences and especially by the DNA sequence encoding BDNF.

Preferably, the defective virus of the invention conserves the sequences in its genome which are necessary for the encapsulation of the viral particles. Still more preferably, as indicated above, the genome of the defective recombinant virus according to the invention comprises the ITR sequences, a sequence allowing encapsulation, the non-functional E1 gene and at least one non-functional E2, E4 or L1-L5 gene.

There are various adenovirus serotypes, whose structure and properties vary somewhat. Among these serotypes, the use of the type 2 or 5 human adenoviruses (Ad 2 or Ad 5) or of adenoviruses of animal origin (see Application FR 93 05954) is preferred within the framework of the present invention. Among the adenoviruses of animal origin which can be used within the framework of the present invention, there may be mentioned adenoviruses of canine, bovine, murine (Example: Mavl, Beard et al., Virology 75 (1990) 81), ovine, procine, avian or alternatively simian (Example: SAV) origin. Preferably, the adenovirus of animal origin is a canine adenovirus, or, more preferably, a CAV2 adenovirus [Manhattan strain or A26/61 (ATCC VR-800) for example]. Preferably, adenoviruses of human or canine or mixed origin are used within the framework of the invention.

The defective recombinant adenoviruses according to the invention can be prepared by any technique known to a person skilled in the art (Levrero et al., Gene 101 (1991) 195, EP 185 573; Graham, EMBO J. 3 (1984) 2917). In particular, they can be prepared by homologous recombination between an adenovirus and a plasmid carrying, inter alia, the DNA sequence encoding BDNF. The homologous recombination occurs after co-transfection of the said adenoviruses and plasmid into an appropriate cell line. The cell line used should preferably (i) be transformable by the said elements, and (ii) contain the sequences capable of complementing the defective adenovirus genome part, preferably in integrated form in order to avoid risks of recombination. As an example of a cell line, there may be mentioned the human embryonic kidney line 293 (Graham et al., J. Gen. Virol. 36 (1977) 59) which contains especially, integrated in its genome, the left hand part of the genome of an Ad5 adenovirus (12%). Strategies for constructing vectors derived from adenoviruses have also been described in Applications Nos. FR 93 05954 and FR 93 08596 which are incorporated herein by way of reference.

Next, the adenoviruses which have multiplied are recovered and purified according to conventional molecular biology techniques as illustrated in the examples.

As indicated above, the present invention also relates to any use of an adenovirus as described above for the preparation of a pharmaceutical composition intended for the treatment and/or prevention of neurodegenerative diseases. More particularly, it relates to any use of these adenoviruses for the preparation of a pharmaceutical composition intended for the treatment and/or prevention of Parkinson's disease, Alzheimer's disease, Amyotrophic Lateral Sclerosis (ALS), Huntington's disease, epilepsy and vascular dementia.

The present invention also relates to a pharmaceutical composition containing one or more defective recombinant adenoviruses as described above. These pharmaceutical compositions can be formulated for topical, oral, parenteral, intranasal, intravenous, intramuscular, subcutaneous, intraocular or transdermal administration and the like. Preferably, the pharmaceutical compositions of the invention contain a pharmaceutically acceptable vehicle for an injectable formulation, especially for a direct injection into the patient's nervous system. This may be in particular isotonic sterile solutions, or dry, especially freeze-dried, compositions which, upon addition, depending on the case, of sterilized water or physiological saline, permit the preparation of injectable solutions. Direct injection into the patient's nervous system is advantageous since it makes it possible to concentrate the therapeutic effect at the level of the affected tissues. Direct injection into the patient's central nervous system is advantageously carried out by means of a stereotaxic injection apparatus. The use of such an apparatus makes it possible, indeed, to target the injection site with great precision.

In this respect, the invention also relates to a method for treating neurodegenerative diseases comprising the administration to a patient of a recombinant adenovirus as defined above. More particularly, the invention relates to the method for treating neurodegenerative diseases comprising the stereotaxic administration of a recombinant adenovirus as defined above.

The doses of defective recombinant adenovirus used for the injection can be adjusted according to various parameters, especially according to the mode of administration used, the pathology concerned, or alternatively the desired duration of treatment. Generally, the recombinant adenoviruses according to the invention are formulated and administered in the form of doses of between 10⁴ and 10¹⁴ pfu/ml, and preferably 10⁶ to 10¹⁰ pfu/ml. The term pfu (plaque forming unit) corresponds to the infectivity of a virus solution, and is determined by infecting an appropriate cell culture, and then measuring, generally after 48 hours, the number of plaques of infected cells. The techniques for determining the pfu titre of a viral solution are well documented in the literature.

Another subject of the invention relates to any mammalian cell infected by one or more defective recombinant adenoviruses as described above. More particularly, the invention relates to any human cell population infected by these adenoviruses. They may be in particular fibroblasts, myoblasts, hepatocytes, keratinocytes, endothelial cells, glial cells and the like.

The cells according to the invention can be obtained from primary cultures. They can be collected by any technique known to a person skilled in the art and then cultured under conditions permitting their proliferation. As regards more particularly fibroblasts, these can be easily obtained from biopsies, for example according to the technique described by Ham [Methods Cell. Biol. 21a (1980) 255]. These cells can be used directly for infection by the adenoviruses, or preserved, for example by freezing, for establishing autologous libraries, for subsequent use. The cells according to the invention may also be secondary cultures obtained for example from pre-established libraries.

The cultured cells are then infected with recombinant adenoviruses, so as to confer on them the capacity to produce BDNF. The infection is carried out in vitro according to techniques known to persons skilled in the art. In particular, according to the type of cells us d and desired number of virus copies per cell, persons skilled in the art can adjust the multiplicity of infection and optionally the number of cycles of infection performed. It is clearly understood that these steps should be carried out under appropriate sterile conditions when the cells are intended for administration in vivo. The recombinant adenovirus doses used for the infection of the cells can be adjusted by persons skilled in the art according to the desired aim. The conditions described above for the administration in vivo can be applied to infection in vitro.

Another subject of the invention relates to an implant comprising mammalian cells infected with one or more defective recombinant adenoviruses as described above, and an extracellular matrix. Preferably, the implants according to the invention comprise 10⁵ to 10¹⁰ cells. More preferably, they comprise 106 to 108.

More particularly, in the implants of the invention, the extracellular matrix comprises a gelling compound and optionally a support permitting anchorage of the cells.

For the preparation of the implant according to the invention, various types of gelling agents can be used. The gelling agents are used for the inclusion of the cells in a matrix having the constitution of a gel, and to enhance the anchorage of the cells on the support, where appropriate. Various cell adhesion agents can therefore be used as gelling agents, such as especially collagen, gelatin, glycosaminoglycans, fibronectin, lectins, and the like. Preferably, collagen is used in the framework of the present invention. This may be collagen of human, bovine or murine origin. More preferably, type I collagen is used.

As indicated above, the compositions according to the invention advantageously comprise a support by permitting anchorage of the cells. The term anchorage designates any form of biological and/or chemical and/or physical interaction resulting in the adhesion and/or binding of the cells on to the support. Moreover, the cells can either cover the support used, or penetrate inside this support, or both. The use of a solid, non-toxic and/or biocompatible support is preferred within the framework of the invention. In particular, it is possible to use polytetra-fluoroethylene (PTFE) fibres or a support of biological origin.

The implants according to the invention can be implanted at different sites in the body. In particular, the implantation can be carried out in the peritoneal cavity, in the subcutaneous tissue (suprapubian region, iliac and inguinal fossae, and the like), in an organ, a muscle, a tumour, the central nervous system or alternatively under a mucous membrane. The implants according to the invention are particularly advantageous in the sense that they make it possible to control the release of the therapeutic product in the body: this release is first determined by the multiplicity of infection and by the number of implanted cells. Next, the release can be controlled either by the removal of the implant, which permanently stops the treatment, or by the use of regulable expression systems, which make it possible to induce or to repress the expression of the therapeutic genes.

The present invention thus offers a very effective means for the treatment or prevention of neurodegenerative diseases. It is most particularly adapted to the treatment of Alzheimer's, Parkinson's, Huntington's or ALS diseases. The adenoviral vectors according to the invention have, in addition, substantial advantages linked especially to their very is high efficiency of infection of the nerve cells, which makes it possible to carry out infections using small volumes of viral suspension. Furthermore, the infection by the adenoviruses of the invention is highly localized at the site of injection which avoids the risks of diffusion to the neighbouring cerebral structures.

In addition, this treatment may apply both to man and to any animal such as ovines, bovines, domestic animals (dogs, cats and the like), horses, fish and the like.

The present invention will be more completely described with the aid of the following examples which should be considered as illustrative and non-limiting.

### LEGEND TO THE FIGURES

FIG. 1: Representation of the vector pXL2244

FIG. 2: Representation of the vector pSh-Ad-BDNF.

### GENERAL MOLECULAR BIOLOGY TECHNIQUES

The methods conventionally used in molecular biology, such as preparative extractions of plasmid DNA, centrifugation of plasmid DNA in caesium chloride gradient, agarose or acrylamide gel electrophoresis, purification of DNA fragments by electroelution, phenol or phenol-chloroform extraction of proteins, ethanol or isopropanol precipitation of DNA in saline medium, transformation in Escherichia coli and the like, are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Prococols in. Molecular Biology”, John Wiley & Sons, New York, 1987].

The pBR322- and pUC- type plasmids and the phages of the M13 series are of commercial origin (Bethesda Research Laboratories).

For the ligations, the DNA fragments can be separated according to their size by agarose or acrylamide gel electrophoresis, extracted with phenol or with a phenol/chloroform mixture, precipitated with ethanol and then incubated in the presence of phage T4 DNA ligase (Biolabs) according to the recommendations of the supplier.

The filling of the protruding 5′ ends can be performed with the Klenow fragment of E. coli DNA polymerase I (Biolabs) according to the specifications of the supplier. The destruction of the protruding 3′ ends is performed in the presence of phage T4 DNA polymerase (Biolabs) used according to the recommendations of the manufacturer. The destruction of the protruding 5′ ends is performed by a controlled treatment with S1 nuclease.

Site-directed mutagenesis in vitro by synthetic oligodeoxynucleotides can be performed according to the method developed by Taylor et al. [Nucleic Acids Res. 13 (1985) 8749-8764] using the kit distributed by Amersham.

The enzymatic amplification of the DNA fragments by the so-called PCR technique [Polymerase-catalyzed Chain Reaction, Saiki R. K. et al., Science 230 (1985) 1350-1354; Mullis K. B. and Faloona F. A., Meth. Enzym. 155 (1987) 335-350] can be performed using a DNA thermal cycler (Perkin Elmer Cetus) according to the specifications of the manufacturer.

The verification of the nucleotide sequences can be performed by the method developed by Sanger et al. [Proc. Natl. Acad. Sci. USA, 74 (1977) 5463-5467] using the kit distributed by Amersham.

### EXAMPLES

### Example 1

### Construction of the Vector pSh-Ad-BDNF

This example describes the construction of a vector comprising a DNA sequence encoding BDNF under the control of a promoter consisting of the Rous sarcoma virus LTR (RSV-LTR).

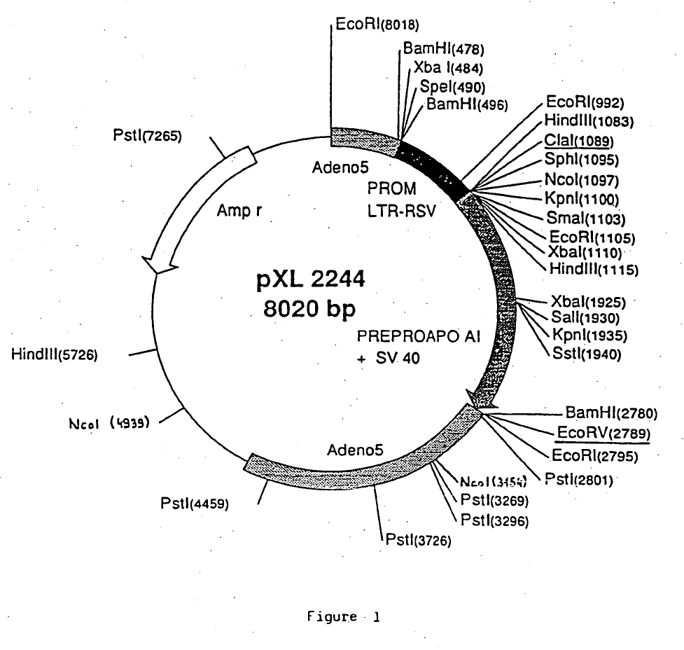

1.1. Starting vector (pXL2244): The plasmid pXL2244 contains the ApoAI cDNA under the control of the RSV virus LTR promoter, as well as the Ad5 adenovirus sequences (FIG. 1). It was constructed by inserting a ClaI-EcoRV fragment containing the cDNA encoding preproApoAI into the vector pLTR RSV-βgal (Stratford-Perricaudet et al., J. Clin. Invest. 90 (1992) 626), digested with the same enzymes.

1.2. Cloning of a cDNA encoding prepro-BDNF. The complete cDNA encoding rat prepro-BDNF (790 pb) was cloned from a rat genomic DNA library by the PCR technique, using as primer the following oligonucleotides:

| | |
|---|---|
| 5′ oligonucleotide: | |
| 5′-TTCATCGAATTCCACCAGGTGAGAAG-3′ | (SEQ ID No. 1) |
| 3′ oligonucleotide: | |
| 5′-AATATAATCTAGACAACATAAATCC-3′ | (SEQ ID No. 2) |

The 5′ region of the sequence obtained was then modified by insertion of a ClaI restriction site, 25 bases upstream of ATG. This site was introduced by PCR by means of the following oligonucleotides:

| | |
|---|---|
| 5′ oligonucleotide: | |
| 5′-TAGCTTCATCGATTTCCACCAG-3′ | (SEQ ID No. 3) |
| 3′ oligonucleotide: | |
| 5′-AATATAATCTAGACAACATAAATCC-3′ | (SEQ ID No. 4) |

The sequence thus obtained was then subcloned into the plasmid pCRII (Invitrogen) to generate the plasmid pCRII-BDNF.

1.3. Construction of the Vector pSh-Ad-BDNF

This example describes the construction of the vector pSh-Ad-BDNF containing, under the control of the RSV virus LTR, the sequence encoding prepro-BDNF as well as Ad5 adenovirus sequences permitting the recombination in vivo.

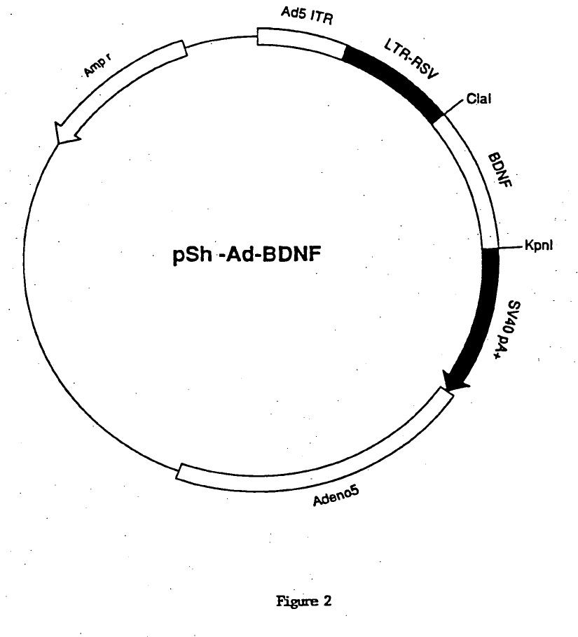

The vector pCR11-BDNF was digested with the enzymes ClaI and KpnI, and the resulting 0.85 kb fragment, containing the sequence encoding prepro-BDNF, was then isolated and purified by LMP (Low Melting Point) agarose gel electrophoresis. In parallel, the vector pXL2244 was digested with the same ClaI and KpnI restriction enzymes, and then precipitated after inactivation of the latter. The resulting linear vector, previously isolated and purified by agarose gel electrophoresis, and the 0.85 kb fragment were then ligated in order to generate the vector pSh-Ad-BDNF (FIG. 2).

### Example 2

### Construction of the Vector pSh-Ad-BDNFtag

This example describes the construction of a second vector comprising a fusion DNA sequence encoding BDNF under the control of a promoter consisting of the Rous sarcoma virus LTR (RSV-LTR).

2.1. Generation of the fusion DNA: In this example, an alternative form of the DNA sequence encoding prepro-BDNF was constructed. This form was obtained by insertion, at the terminal 3′ end of the sequence described in Example 1.2., of a sequence encoding an epitope of seven amino acids (tag) recognized by a commercially available antibody (IBI, Integra Biosciences, Eaubonne, France). The sequence of the region thus fused is the following (SEQ ID No. 5):

Arg Gly Asp Tyr Lys Asp Asp Asp Asp ***
AGA GGC GAC TAC AAG GAC GAC GAT GAC TAG
-------
C-ter fused seq.
BDNF

The sequence thus obtained was then subcloned into the plasmid pCRII (Invitrogen) to generate the plasmid pCRII-BDNFtag.

2.2. Construction of the Vector pSh-Ad-BDNFtag

This example describes the construction of the vector pSh-Ad-BDNFtag containing the fusion sequence encoding prepro-BDNF, under the control of the RSV virus LTR, as well as the Ad5 adenovirus sequences permitting the recombination in vivo.

The vector pCRII-BDNFtag was digested with the enzymes ClaI and KpnI, and the resulting 0.87 kb fragment, containing the sequence encoding prepro-BDNFtag was then isolated and purified by LMP (Low Melting Point) agarose gel electrophoresis. In parallel, vector pXL2244 (Example 1.1.) was digested with the same ClaI and KpnI restriction enzymes, and then precipitated after inactivation of the latter. The resulting linear vector, previously isolated- and purified by agarose gel electrophoresis, and the 0.87 kb fragment were then ligated so as to generate the vector pSh-Ad-BDNFtag.

### Example 3

### Functionality of the Vectors pSh-Ad-BDNF and pSh-Ad-BDNFtag

The capacity of the vectors pSh-Ad-BDNF and pSh-Ad-BDNFtag to express in cell culture a biologically active-form of BDNF was demonstrated by transient transfection of COS1 cells. For that, the cells (2×10⁶ cells per dish 10 cm in diameter) were transfected (8 μg of vector) in the presence of Transfectam. After 48 hours, the cell culture supernatant was harvested. Serial dilutions (1/200 and 1/50) of this supernatant were then added to primary cultures of septum neurons (Hefti et al. In Dissection and Tissue cultures: Manual of the Nervous System (1989) 172, Alan R. Liss, Inc). Trophic effect (cell survival and neuritic growth) on these cultures was observed after staining, and the differentiator effect by assaying the expression of the choline acetyl transferase enzyme (CHAT) according to the technique described by Formum (J. Neurochem. 24 (1975) 407).

### Example 4

### Construction of Recombinant Adenoviruses Containing a Sequence Encoding BDNF

4.1. Construction of the Adenovirus Ad-BDNF

The vector pSh-Ad-BDNF was linearized and cotransfected with a deficient adenoviral vector, into the helper cells (line 293) providing in trans the functions encoded by the adenovirus E1 regions (E1A and E1B).

More specifically, the adenovirus Ad-BDNF was obtained by homologous recombination in-vivo between the mutant adenovirus Ad-dl1324 (Thimmappaya et al., Cell 31 (1982) 543) and the vector pSh-Ad-BDNF, according to the following procedure: the plasmid pSh-Ad-BDNF and the adenovirus Ad-dl1324, linearized with the enzyme ClaI, were cotransfected into line 293 in the presence of calcium phosphate, so as to allow the homologous recombination. The recombinant adenoviruses thus generated were selected by plaque purification. After isolation, the recombinant adenovirus DNA was amplified in the cell line 293, which gives a culture supernatant containing the unpurified recombinant defective adenovirus having a titre of about 10¹⁰ pfu/ml.

The viral particles were then purified by caesium chloride gradient centrifugation according to known techniques (see especially Graham et al., Virology 52 (1973) 456). The adenovirus Ad-BDNF can be preserved at −80° C. in 20% glycerol.

4.2. Construction of the Adenovirus Ad-BDNFtag

The adenovirus Ad-BDNFtag was constructed according to the same procedure as the adenovirus Ad-BDNF, but using as starting vector the vector pSh-Ad-BDNFtag.

### Example 5

### Functionality of the Adenovirus Ad-BDNF

The capacity of the adenovirus Ad-BDNF to infect cultured cells and to express in the culture medium a biologically active form of BDNF is demonstrated by infecting human 293 and rat PC12 lines. The presence of active BDNF in the culture supernatant was then determined under the same conditions as in Example 3.

These studies make it possible to demonstrate that the adenovirus does indeed express a biologically active form of BDNF in cell culture.

### Example 6

### Transfer in vivo of the BDNF Gene by a Recombinant Adenovirus to Rats with Lesion of the Fimbria-formix

This example describes the transfer of the BDNF gene in vivo by means of an adenoviral vector according to the invention. It shows, on an animal model of lesion of the fimbria-formix, that the vectors of the invention make it possible to induce the expression in vivo of therapeutic quantities of BDNF.

In previously anaesthetized rats, the septo-hippocampal route (fimbria-formix) was sectioned at the level of the left hemisphere. This mechanical lesion was made with the aid of a retractable surgical knife. The stereotaxic coordinates used to this effect are, relative to the bregma: AP: −1.7; ML: +1.5; V: −5.5 to −0.5.

The BDNF recombinant adenovirus was injected immediately after the lesion, into the median nucleus of the septum and into the dorsal part of the deafferentated hippocampus (hippocampus on the lesion side). More particularly, the injected adenovirus is the adenovirus Ad-BDNF prepared in Example 4.1., used in purified form (3.5×10⁶ pfu/μl), in a phosphate buffered saline solution (PBS).

The injections are carried out with the aid of a cannula (external diameter 280 μm) connected to a pump. The rate of injection is fixed at 0.5 μl/min, after which, the cannula remains in place for 4 additional minutes before being withdrawn. The volumes of injection into the hippocampus and the septum are respectively 3 μl and 2 μl. The adenovirus concentration injected is 3.5×10⁶ pfu/μl.

For injection into the hippocampus, the stereotaxic coordinates are the following: AP=−4; ML=3.5; V=−3.1 (the AP and ML coordinates are determined relative to the bregma, the V coordinate relative to the surface of the cranial bone at the level of the bregma.

For the injection into the septum, the stereotaxic coordinates are the following: AP=0.1; ML=1; V=−6 (the AP and ML coordinates are determined relative to the bregma, the V coordinte relative to the surface of the cranial bone at the level of the bregma. Under this condition, the cannula is at an angle of 9 degrees relative to the vertical (in the mediolateral direction) in order to avoid the median venous sinus.

The therapeutic effects of the administration of the adenovirus according to the inventor have been demonstrated by three types of analysis: a histological and immunohistochemical analysis, a quantitative analysis and a behavioural analysis.

Histological Immunohistochemical Analysis

The mechanical lesion of the fimbria-formix induces a loss of cholinergic neurons (visualized in immunohistology by an anti-choline acetyl transferase, CHAT, antibody) in the median septum, as well as cholinergic denervation in the hippocampus (detected in histochemistry by the acetyl choline esterase, AChE, activity).

Histological analysis of the injected brains is carried out 3 weeks after the intracerebral injection of the adenovirus Ad-BDNF. For that, the animals are sacrificed, under anaesthesia, by intracardiac infusion of 4% paraformaldehyde. After removal, postfixing and cryoprotection, the brain is sectioned using a cryomat along the coronal plane: coronal serial sections 30 μm thick are made over the entire length of the median septum and in the anterior, median and posterior regions of the hippocampus. For the median septum, sections 180 μm apart (1 section out of 6) are stained with cresyl violet (in order to evaluate the neuronal density) and immunolabelled with an anti-ChAT antibody (Biochem) (so as to identify the cholinergic neurons). The immunohistochemical method is that of streptavidin-biotin peroxidase visualized with DAB. For the hippocampus, sections 180 μm apart are stained according to the histochemical method for AChE (acetyl choline esterase) so as to detect the cholinergic innervation. The sections are mounted on glass slides.

Ouantitative Analysis

The number of cholinergic neurons (ChAT-positive), in the median septum is the parameter for evaluation of the effects of the adenovirus Ad-BDNF. The enumeration is carried out on a sample (1 section out of 6 over the entire length of the median septum). For each section, the ChAT-positive neurons are counted separately on both sides of the septum. The cumulative results for all the sections are expressed by the ratio of the number of ChAT-positive neurons on the injured side over the number of ChAT-positive neurons on the uninjured side.

Behavioural Analysis

It is known that a bilateral lesion of the septo-hippocampal route leads to memory deficiency. In order to evaluate the protective functional effects of an injection of adenovirus Ad-BDNF on this type of lesion, the memory performances of the animals were analysed during 2 behavioural tests: the Morris swimming pool test (visuospatial reference memory) and the TMTT test (two-trials memory task; “short-term memory of a new environment”).

### Example 7

### Transfer in vivo of the BDNF Gene by a Recombinant Adenovirus to Rats with Lesion of the Nigro-striatal Route

This example describes the transfer of the BDNF gene in vivo by means of an adenoviral vector according to the invention. It shows, in an animal model of the lesion of the nigro-striatal route, that the vectors of the invention make it possible to induce the expression in vivo of thereapeutic quantities of BDNF.

In previously anaesthetized rats, the nigro-striatal route was injured at the level of the median mesencephalic bundle (MFB) by injection of the toxin 6-hydroxydopamine (60H-DA). This chemical lesion by injection was unilateral along the following stereotaxic coordinates: AP: 0 and −1; ML: +1.6; V: −8.6 and −9 (the AP and ML coordinates are determined relative to the bregma, the V coordinate relative to the dura mater). The incisive bar is fixed at the +5 mm level.

The recombinant adenovirus BDNF was injected immediately after the lesion, into the black substance and the striatum, on the lesion side. More particularly, the injected adenovirus is the adenovirus Ad-BDNF prepared in Example 4.1., used in purified form (3.5×10⁶ pfu/μl), in a phosphate buffered saline solution (PBS).

The injections were carried out with the aid of a cannula (external diameter 280 μm) connected to a pump. The rate of injection is fixed at 0.5 μl/min, after which the cannula remains in place for an additional 4 minutes before being withdrawn. The injection volumes into the striatum and the black substance are 2×3 μl and 2 μl respectively. The adenovirus concentration injected is 3.5×10⁶ pfu/μl.

For the injection into the black substance, the stereotaxic coordinates are the following:

AP=−5.8, ML=+2; V=−7.5 (the AP and ML coordinates are determined relative to the bregma, the V coordinate relative to the dura mater).

For the injections into the striatum, the stereotaxic coordinates are the following: AP=+0.5 and −0.5; ML=3; V=−5.5 (the AP and ML coordinates are determined relative to the bregma, the V coordinate relative to the dura mater).

The therapeutic effects of the administration of the adenovirus according to the invention were detected by three types of analysis: a histological and immunohistochemical analysis, a quantitative analysis and a behavioural analysis.

Histological and Immunohistochemical Analysis

Chemical lesion of the nigro-striatal route induces neuronal loss in the black substance as well as the dopaminergic denervation in the striatum (visualized in immunohistology by an anti-tyrosine hydroxylase, TH, antibody).

Histological analysis of the injected brains is carried out 3 weeks after the intracerebral injection of the adenovirus Ad-BDNF under the conditions described in Example 6. The coronal serial sections 30 μm thick are made in the black substance and the striatum. Sections 180 μm apart (1 section out of 6) are stained with cresyl violet (so as to evaluate the neuronal density) and immunolabelled with an anti-tyrosine hydroxylase (TH) antibody (so as to detect the dopaminergic neurons in the black substance and their innervation in the striatum).

Ouantitative Analysis

The number of dopaminergic neurons (TH-positive) in the black substance is the parameter for evaluation of the effects of the adenovirus Ad-BDNF. The enumeration is carried out on a sample (1 section out of 6 over the entire length of the black substance). For each section, the TH-positive neurons are counted separately on both sides of the black substance. The cumulative results for all the sections are expressed as a proportion: number of TH-positive neurons on the injured side relative to the number of TH-positive neurons on the uninjured side.

Behavioural Analysis

In order to evaluate the protective functional effects of an injection of adenovirus Ad-BDNF on the lesion of the nigro-striatal route, the sensorimotor performances of the animals were analysed during 2 behavioural tests: the tests of rotation induced by dopaminergic agonists (apomorphine, amphetamine and levodopa), and the prehension (paw-reaching) test.

### Example 8

### Transfer in vivo of the BDNF Gene by a Recombinant Adenovirus to Rats with Lesion of the Perforating Route

This example describes the transfer of the BDNF gene in vivo by means of an adenoviral vector according to the invention. It shows, in an animal model of the lesion of the perforating route, that the vectors of the invention make it possible to induce the expression in vivo of therapeutic quantities of BDNF.

In previously anaesthetized rats, the entorhinohippocampal route (perforating route) was unilaterally sectioned with the aid of a surgical knife. The stereotaxic coordinates used to this end are, relative to the lambda: AP: +0.75; ML: +4.1 to 6.6; V: −7.7 (V coordinate determined relative to the dura mater).

The recombinant adenovirus BDNF is injected immediately after the lesion, either at the level of the lesion, or at the level of the hippocampus and the entorhinal cortex. More particularly, the injected adenovirus is the adenovirus Ad-BDNF prepared in Example 4.1., used in purified form (3.5×10⁶ pfu/μl), in a phosphate buffered saline solution (PBS).

The injections were carried out with the aid of a cannula (external diameter 280 μm) connected to a pump. The rate of injection is fixed at 0.5 μl/min, after which the cannula remains in place for an additional 4 minutes before being withdrawn. The injection volumes into the hippocampus, the entorhinal cortex and the lesion site of the perforating route are 3 μl, 2 μl and 2 μl respectively. The adenovirus concentration injected is 3.5×10⁶ pfu/μl.

The therapeutic effects of the administration of the adenovirus according to the invention can be detected by a behavioural analysis under the conditions of Example 6.

1-26. (canceled).

27. A replication defective recombinant adenovirus comprising a DNA sequence cDNA encoding brain-derived neurotrophic factor (BDNF), wherein an adenovirus E1 gene and at least one of adenovirus E2, E4 or L1-L5 genes are non-functional, and wherein the BDNF encoding cDNA is operably linked to a signal controlling expression in a cell of the central nervous system.

28. The replication defective recombinant adenovirus according to claim 27, wherein the cDNA encodes prepro-BDNF.

29. (canceled).

30. (canceled).

31. The replication defective recombinant adenovirus according to claim 27, wherein the cDNA encodes human prepro-BDNF.

32. The replication defective recombinant adenovirus according to claim 27, wherein the cDNA is operably linked to a signal controlling expression in a nerve cell.

33. The replication defective recombinant adenovirus according to claim 32, wherein the signal is a viral promoter.

34. The replication defective recombinant adenovirus according to claim 33, wherein the signal is selected from the group consisting of an RSV-LTR promoter, an E1A promoter, a MLP promoter, and a CMV promoter.

35. A replication defective recombinant adenovirus comprising a cDNA encoding human prepro-BDNF, operably linked to an RSV-LTR promoter, wherein an adenovirus E1 gene and at least one of adenovirus E2, E4 or L1-L5 genes are non-functional

36. (canceled).

37. A replication defective recombinant adenovirus comprising a cDNA encoding human brain-derived neurotrophic factor (hBDNF) operably linked to a promoter controlling expression in a nerve cells, wherein an adenovirus E1 gene and at least one of adenovirus E2, E4 or L1-L5 genes are nonfunctional.

38. A replication defective recombinant adenovirus according to claim 37, wherein the promoter is selected from the group consisting of a neuron-specific enolase promoter and a GFAP promoter.

39. (canceled).

40. The replication defective recombinant An adenovirus according to claim 27, comprising ITRs and a sequence permitting encapsulation.

41. The replication defective recombinant adenovirus according to claim 27, wherein the replication defective recombinant adenovirus is a type Ad 2 or Ad 5 human adenovirus or a CAV-2 type canine adenovirus.

42. A method for the treatment and/or prevention of a neurodegenerative disease comprising administration of an effective amount of the replication defective recombinant adenovirus according to claim 27.

43. A method according to claim 42, wherein the neurodegenerative disease is selected from the group consisting of Parkinson's disease, Alzheimer's disease, Huntington's disease and Amyotrophic Lateral Sclerosis (ALS).

44. A pharmaceutical composition comprising the replication defective recombinant adenovirus according to claim 27 and a pharmaceutically acceptable vehicle.

45. The pharmaceutical composition according to claim 44, in injectable form.

46. The pharmaceutical composition according to claim 44, comprising between 10⁴ and 10¹⁴ pfu/ml of replication defective recombinant adenovirus.

47. The pharmaceutical composition according to claim 46, comprising between 10⁶ and 10¹⁰ pfu/ml of replication defective recombinant adenovirus.

48. A mammalian cell infected with the replication defective recombinant adenoviruses according to claim 27.

49. The mammalian cell according to claim 48, wherein the mammalian cell is a human cell.

50. The mammalian cell according to claim 49, wherein the mammalian cell is selected from the group consisting of a fibroblast, a myoblast, a hepatocyte, an endothelial cell, a glial cell and a keratinocyte.

51. An implant comprising a mammalian cells according to claim 48 and an extracellular matrix.

52. The implant according to claim 51, wherein the extracellular matrix comprises a gelling compound selected from the group consisting of collagen, gelatin, glucosaminoglycans, fibronectin and lectins.

53. An implant according to claim 51, wherein the extracellular matrix comprises a support permitting anchorage of the mammalian cells.

54. An implant according to claim 53, wherein the support comprises a polytetrafluoroethylene fiber.


SEQUENCE LISTING (6 sequences)

SEQ ID NO: 1 - DNA (other nucleic acid), 26 bp; single-stranded, linear
  hypothetical: NO
  anti-sense: NO
ttcatcgaattccaccaggtgagaag

SEQ ID NO: 2 - DNA (other nucleic acid), 25 bp; single-stranded, linear
  hypothetical: NO
  anti-sense: NO
aatataatctagacaacataaatcc

SEQ ID NO: 3 - DNA (other nucleic acid), 22 bp; single-stranded, linear
  hypothetical: NO
  anti-sense: NO
tagcttcatcgatttccaccag

SEQ ID NO: 4 - DNA (other nucleic acid), 25 bp; single-stranded, linear
  hypothetical: NO
  anti-sense: NO
aatataatctagacaacataaatcc

SEQ ID NO: 5 - DNA (cDNA), 30 bp; double-stranded, linear
  CDS 1..30
  hypothetical: NO
  anti-sense: NO
agaggcgactacaaggacgacgatgactag

SEQ ID NO: 6 - protein, 9 aa
RGDYKDDDD